(19) United States
(12) Patent Application Publication
(10) Pub. No.: US 2025/0367027 A1
(43) Pub. Date: Dec. 4, 2025

(54) Hypothermia Survival Bag

(71) Applicant: U.S. Army Combat Capabilities Development Command, Soldier Center, APG, MD

(72) Inventors: Kristine D. Isherwood, Natick, MA (US); Chong Whitfield, Natick, MA (US)

(73) Assignee: The United States of America as Represented by the Secretary of the Army, Washington, D.C., (omitted)

(21) Appl. No.: 19/214,095

(22) Filed: May 21, 2025

Related U.S. Application Data
(60) Provisional application No. 63/653,477, filed on May 30, 2024.

(51) Int. Cl. A61F 7/08; A61G 1/048

(52) U.S. Cl. CPC A61F 7/08; A61G 1/048

(57) ABSTRACT

A hypothermia survival bag comprises a waterproof base having a water repellent shell outer layer and waterproof liner, the waterproof base having a bathtub configuration having a head end, a foot end, and left and right sides; an insulated top panel attached to the waterproof base at the foot end, and removably attached to each of the left and right sides with left and right side zippers, wherein the left and right side zippers extend from the foot end to the head end, wherein each of the left and right side zippers includes a plurality of pulls permitting each of the left and right side zippers to be selectively opened in multiple places simultaneously; a foot box; a hood having a brim; a patient card pouch and a plurality of side, head, and foot transport loops attached to the water repellent shell of the waterproof base.

### CROSS-REFERENCE TO RELATED APPLICATIONS

This application claims priority to and the benefit of U.S. Provisional Patent Application Ser. No. 63/653,477, filed 30 May 2024, the disclosure of which is incorporated herein by reference in its entirety.

### FEDERALLY SPONSORED RESEARCH AND DEVELOPMENT

The invention described herein may be manufactured and used by the United States Government for governmental purposes without the payment of any royalty thereon.

### BACKGROUND OF THE INVENTION

Discussions with medical personnel revealed shortcomings with current equipment for treating and transporting hypothermic patients. Current approaches may utilize a simple water barrier, such as a poncho as a bottom layer, with a sleeping pad on top. A first sleep system or sleeping bag is placed on top of the sleeping pad. The hypothermic person may be wrapped with a blanket and placed into a second sleep system. This approach has numerous shortcomings, including highly reduced patient access; patient access at ground level, which results in environmental intrusion and significant heat loss; use of two sleep systems for one patient; ineffective insulation in extreme cold temperatures; relies on external heating to begin heating the patient; does not accommodate the full range of patient statures and girths, especially clothing or equipment that must remain on due to the nature of the injury; does not facilitate movement of a stabilized patient; does not isolate the patient from heat loss to the ground or due to fluid run-off; and can only be used once. What is desired is equipment that aids caregivers in providing care to an exposure- or trauma-induced hypothermic Soldier during extreme cold weather operations.

### SUMMARY OF THE INVENTION

A hypothermia survival bag comprises a waterproof base further comprising a water repellent shell outer layer and a waterproof liner, wherein the waterproof base with insulated walls has a bathtub configuration having a head end, a foot end, and left and right sides; an insulated top panel attached to the waterproof base at the foot end, and removably attached to each of the left and right sides with left and right side zippers, wherein the left and right side zippers extend from the foot end to the head end, wherein each of the left and right side zippers includes a plurality of pulls permitting each of the left and right side zippers to be selectively opened in multiple places simultaneously; a foot box; a hood having a brim; and a patient card pouch attached to an outside of the insulated top panel. The zippers have multiple pulls which makes it possible for medical personnel to access a patient very selectively, without the need to open an entire side of the bag. The bathtub shape of the waterproof base is advantageous in elevating the zippers to the level of the insulated top panel. The elevated zippers aid in keeping snow, rain, groundwater, and other environmental materials out of the survival bag.

According to a first embodiment, the hypothermia survival bag may further include a plurality of side transport loops, a head transport loop, and a foot transport loop attached to the water repellent shell of the waterproof base. It should be emphasized that the side, head, and foot transport loops are not attached to the waterproof liner, but only to the outermost layer, the water repellent shell. The plurality of side transport loops, the head transport loop, and the foot transport loop may be selectively attached to the water repellent shell of the waterproof base in order to accommodate a stabilizing board between the plurality of transport loops and the water repellent shell of the waterproof base.

According to a further embodiment, the insulated top panel may include a duck bill extension at the head end. In a similar manner, the insulated top may include a chest extension to provide extra insulation around the head opening of the hood.

According to another embodiment, the bottom interior of the waterproof base may include a plurality of insulating layers selected from the group consisting of batting with aerogel, a durable water repellent shell, an absorbent pad, and a perforated spacer pad. One or more of the layers may be doubled, if desired, so as to achieve the desired performance goals.

According to a further embodiment, each of the interior left and right sides of the waterproof base may include an insulating curtain attached adjacent the left and right side zippers, the insulating curtain hanging down to the bottom interior of the waterproof base.

According to another embodiment, each of the left and right insulating curtains may include a plurality of insulating layers selected from the group consisting of a batting layer, a batting layer with aerogel, and a metallized shell. One or more of the layers may be doubled, if desired, so as to achieve the desired performance goals.

According to a further embodiment, the insulated top panel may include a plurality of insulating layers selected from the group consisting of batting with aerogel, a wind-resistant shell, and a metallized shell. One or more of the layers may be doubled, if desired, so as to achieve the desired performance goals.

According to another embodiment, the insulating curtains may further comprise insulated extensions to warm the neck and/or feet.

According to a further embodiment, a girth adjustment mechanism may be attached to left and right edges of the insulated top panel. The girth adjustment mechanism may be Y-straps with buckles, and/or a girth adjustment cord laced through eyelets along the left and right edges of the insulated top panel.

According to another embodiment, a length adjustment mechanism may be incorporated into a bottom exterior of the waterproof base. The length adjustment mechanism may include one or more length adjustment cords laced through openings in the side transport loops, the head transport loop, and the foot transport loop.

Components and features described herein may be combined in any desired manner to achieve the desired performance goals.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention. The patent or application file contains at least one drawing executed in color. Copies of this patent or patent application publication with color drawing(s) will be provided by the Office upon request and payment of the necessary fee.

FIGS. 1A-1B present sectional views of a hypothermia survival bag (HSB), according to an embodiment of the invention.

FIGS. 2-4 present elevated perspective views of a hypothermia survival bag (HSB), according to an embodiment of the invention.

FIGS. 5-9 present elevated perspective views of an opened hypothermia survival bag (HSB), from the head end, according to an embodiment of the invention.

FIGS. 10-12 present elevated perspective views of a hypothermia survival bag (HSB), from the foot end, according to an embodiment of the invention.

FIGS. 13-15 present views of the bottom of a hypothermia survival bag (HSB), according to an embodiment of the invention.

FIGS. 16-19 present views of the upper surface of a hypothermia survival bag (HSB), according to an embodiment of the invention.

FIG. 20 presents a view from the foot end of a hypothermia survival bag (HSB), according to an embodiment of the invention.

FIGS. 21-22 present interior views of the hood of a hypothermia survival bag (HSB), according to an embodiment of the invention.

FIGS. 23-24 present an open foot enclosure of a hypothermia survival bag (HSB), according to an embodiment of the invention.

FIGS. 25-27 presents views of a perforated spacer pad for a hypothermia survival bag (HSB), according to an embodiment of the invention.

FIG. 28 presents an arrangement of length adjustment cords for a hypothermia survival bag (HSB), according to an embodiment of the invention.

FIGS. 29-30 present an arrangement of Y-straps and buckles for a hypothermia survival bag (HSB), according to an embodiment of the invention.

FIG. 31 presents neck and chest insulation for a hypothermia survival bag (HSB), according to an embodiment of the invention.

### DETAILED DESCRIPTION OF THE INVENTION

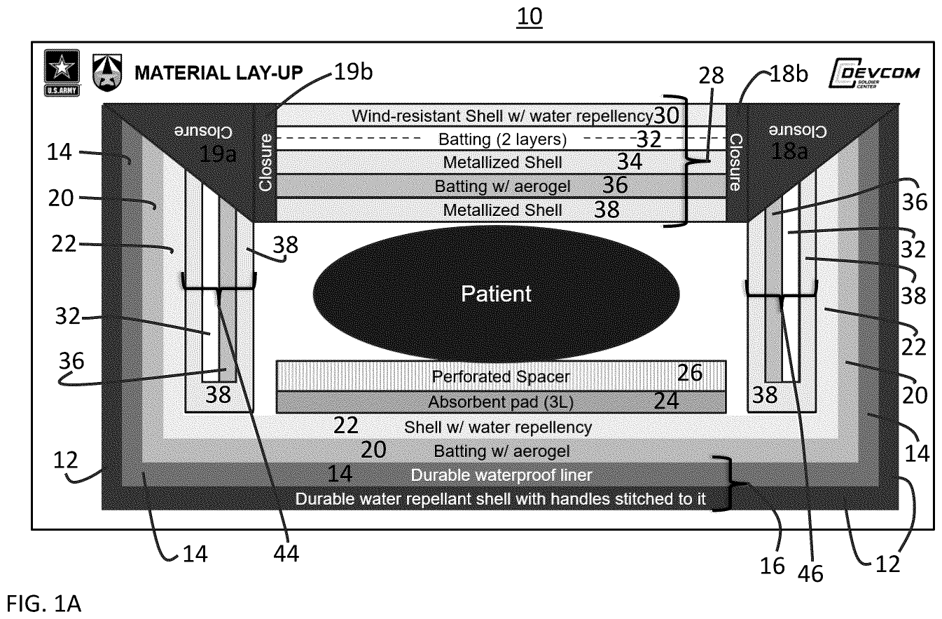

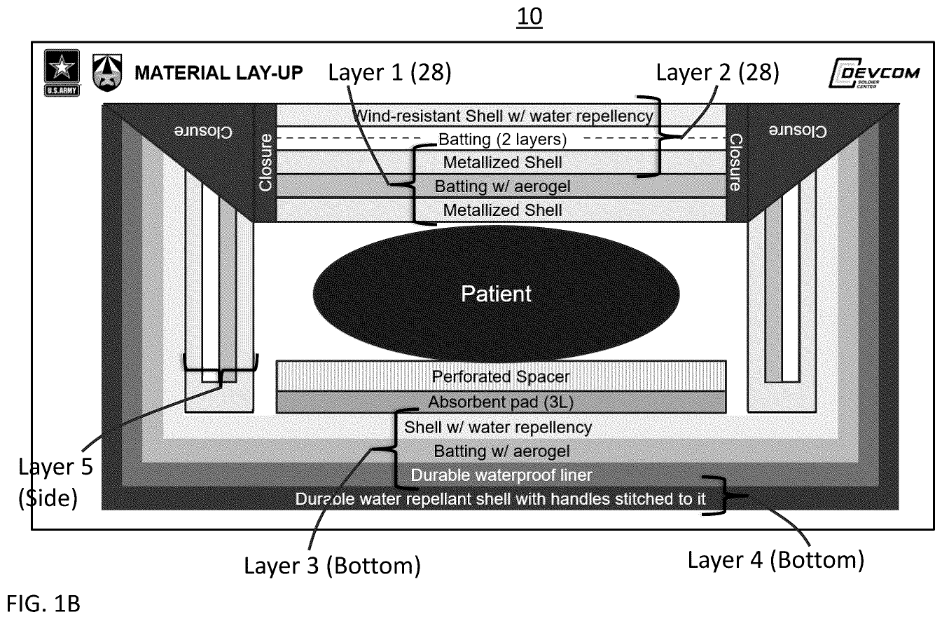

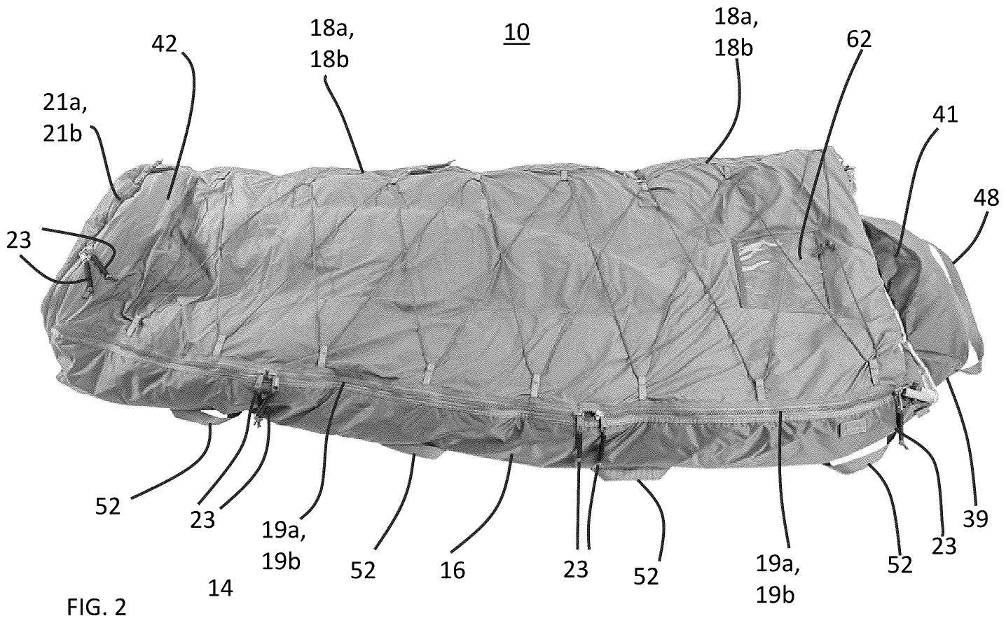

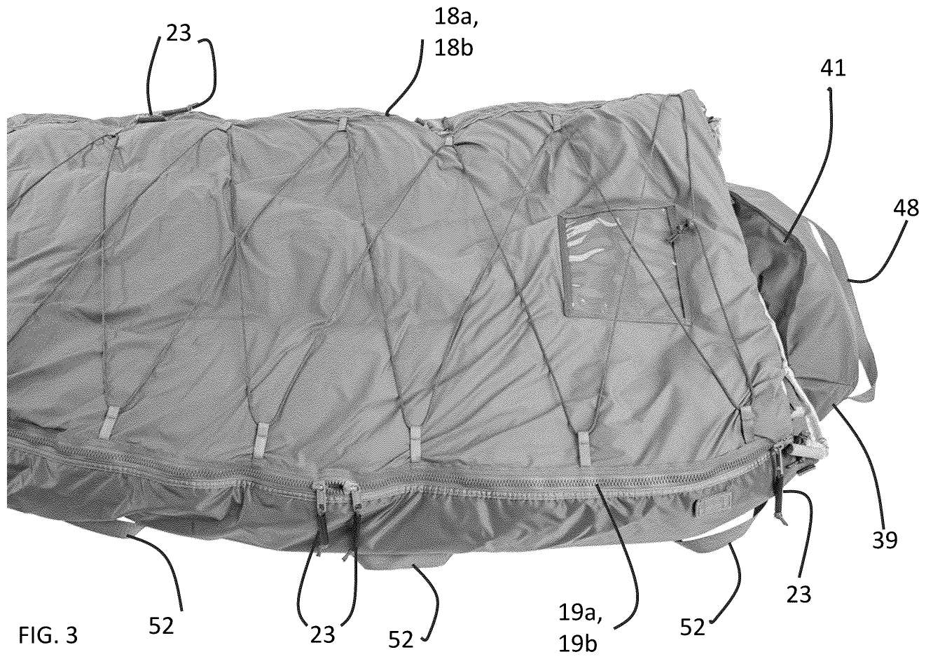

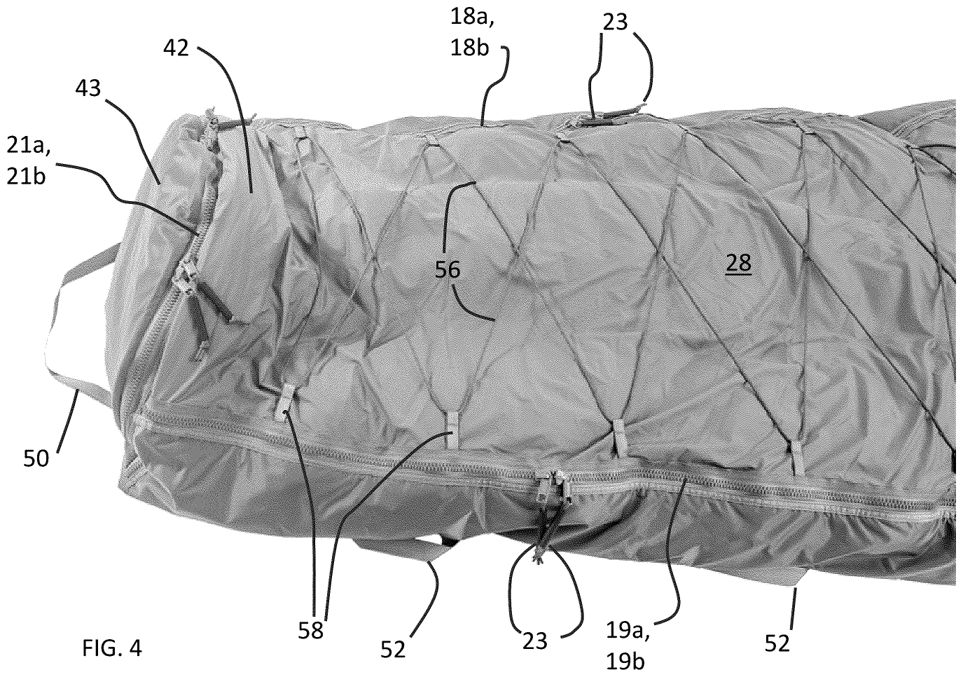

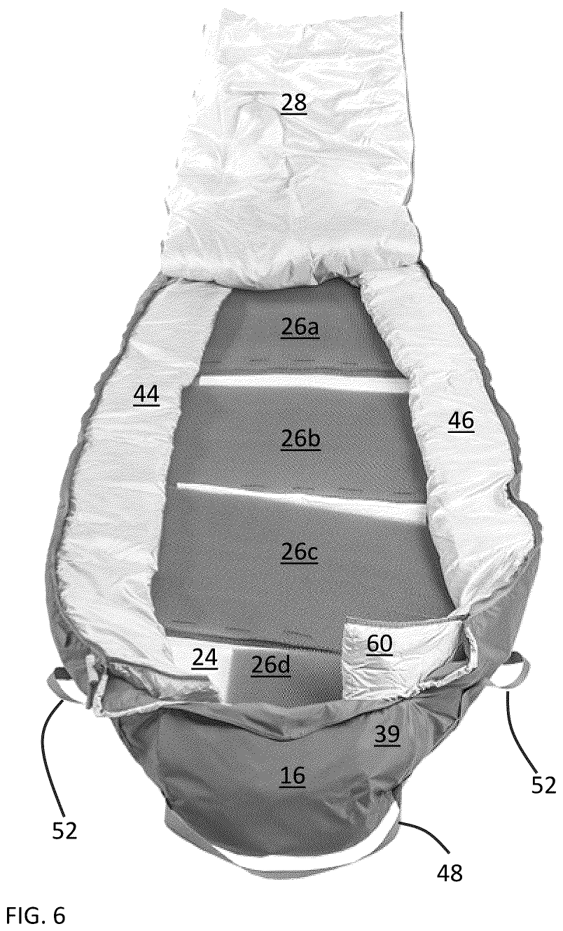

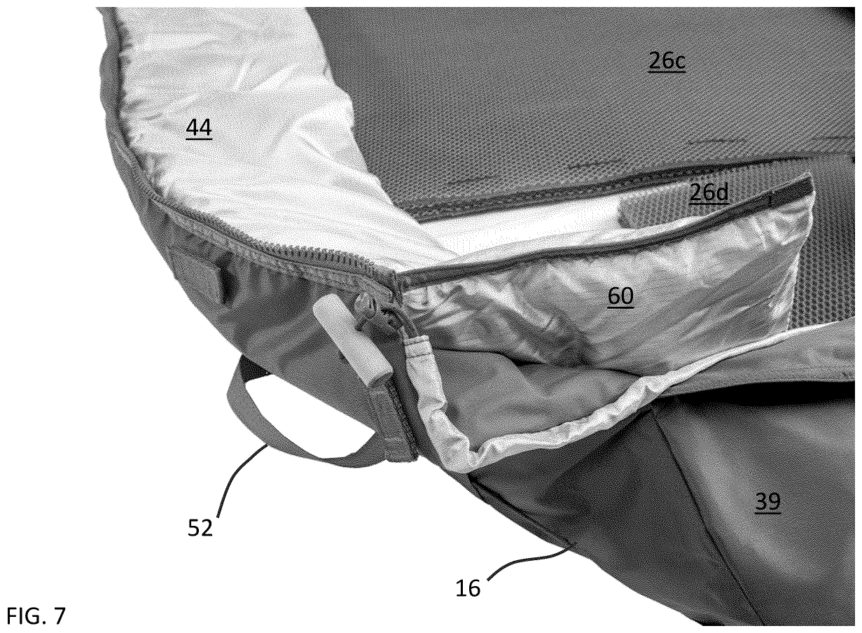

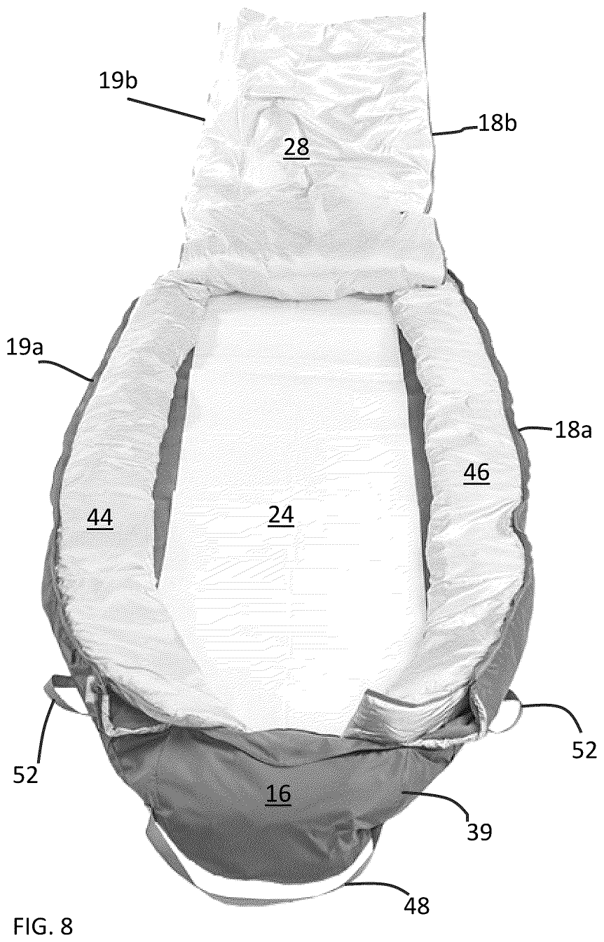

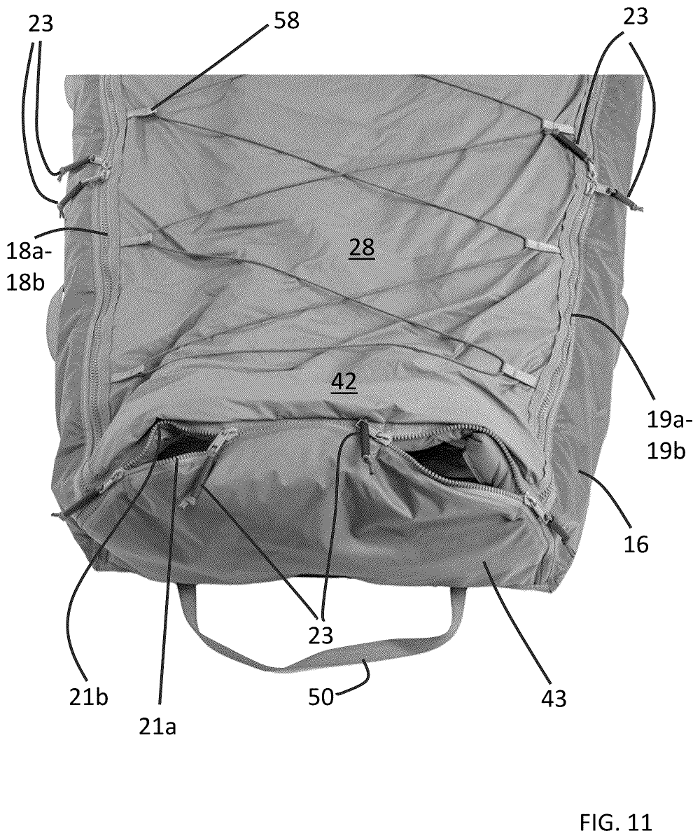

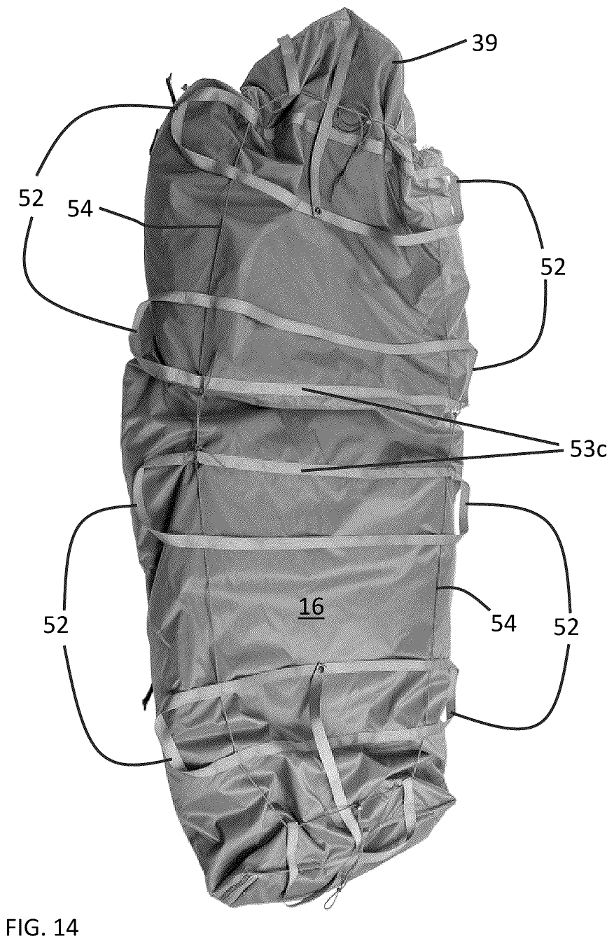

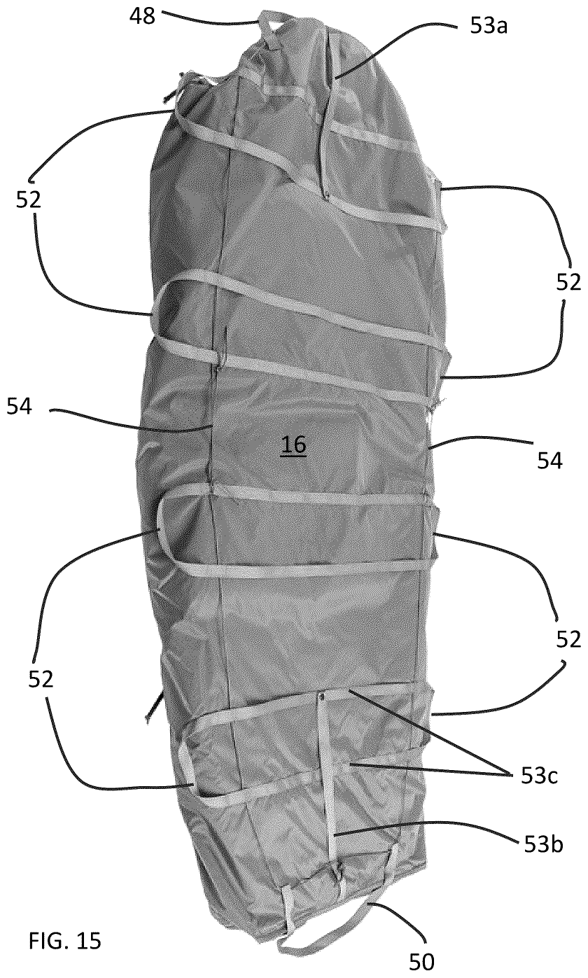

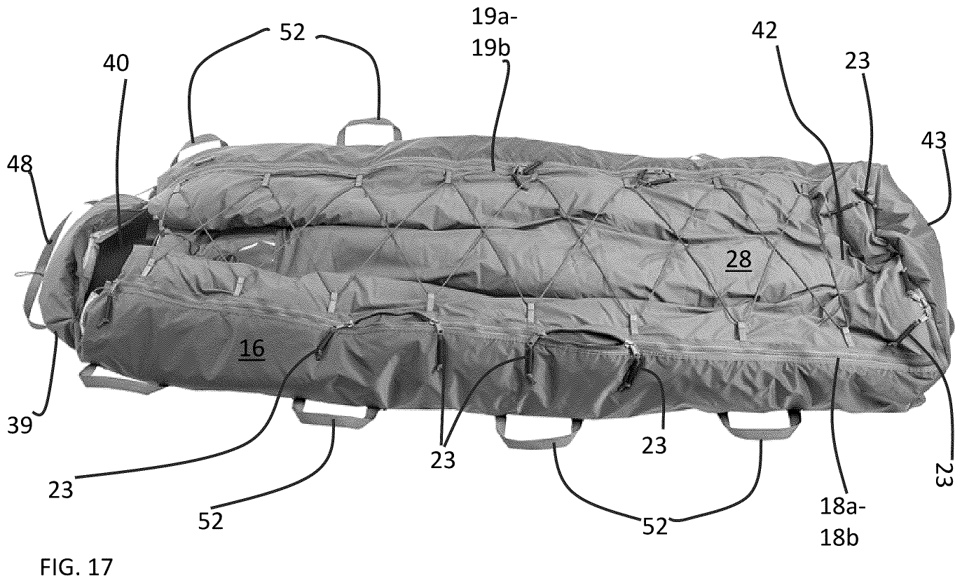

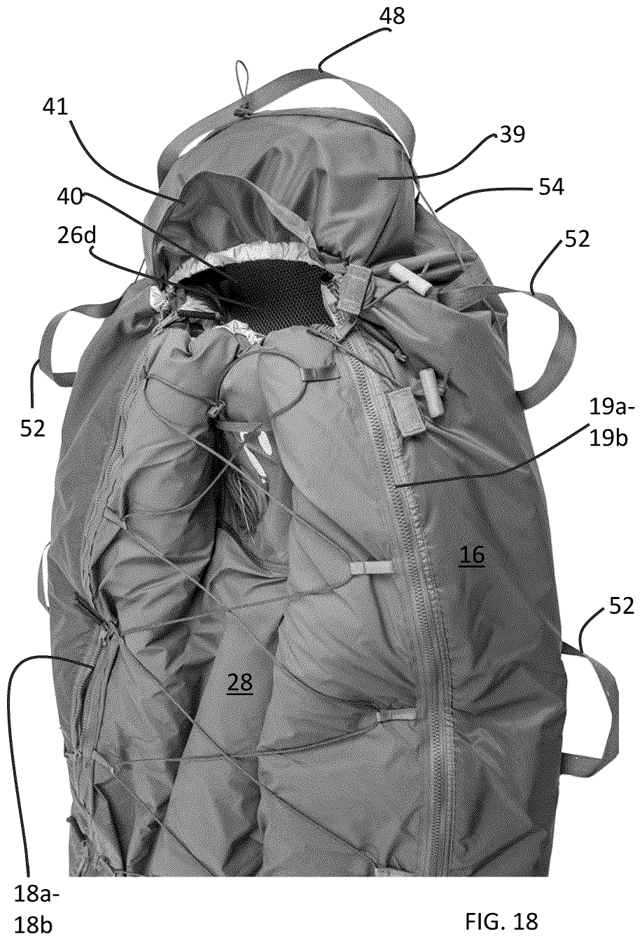

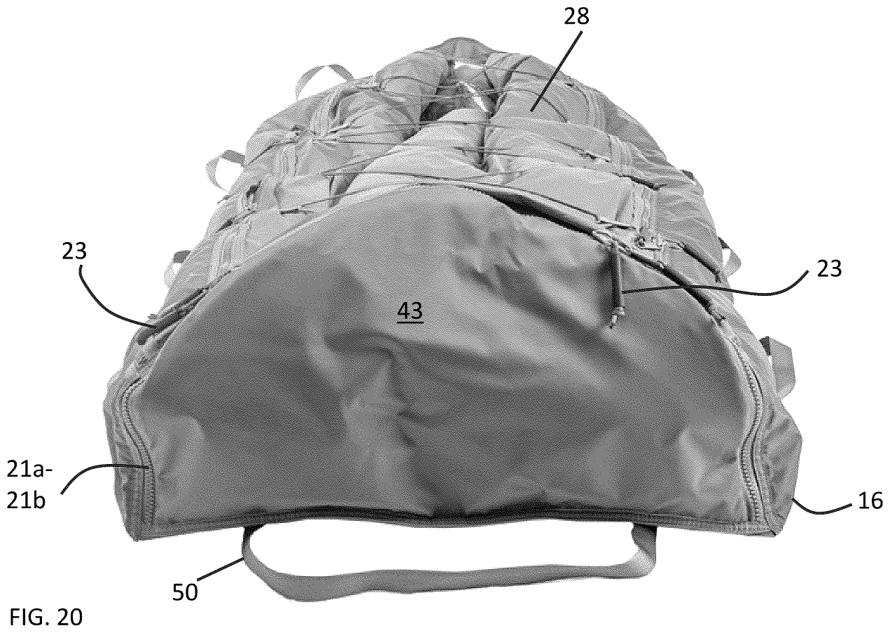

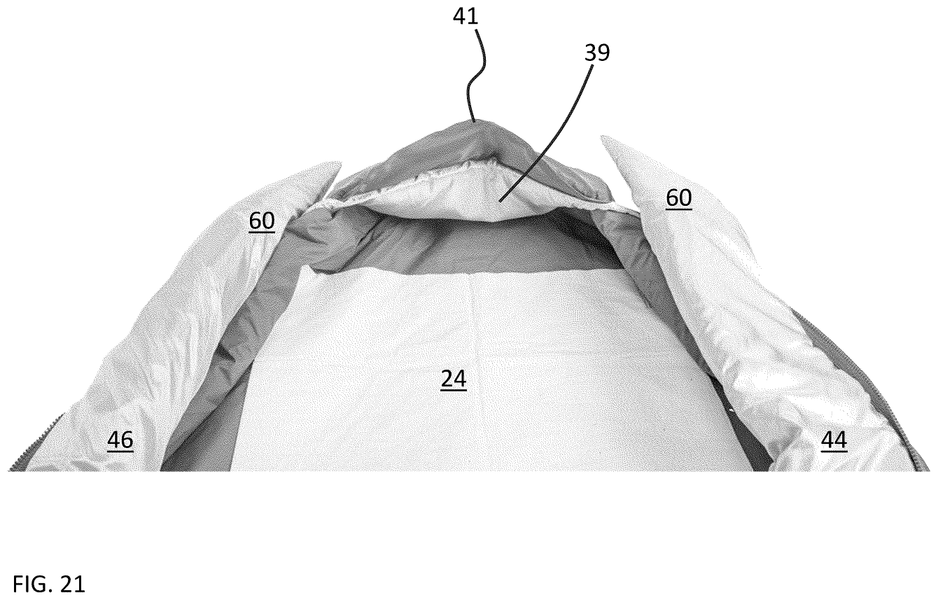

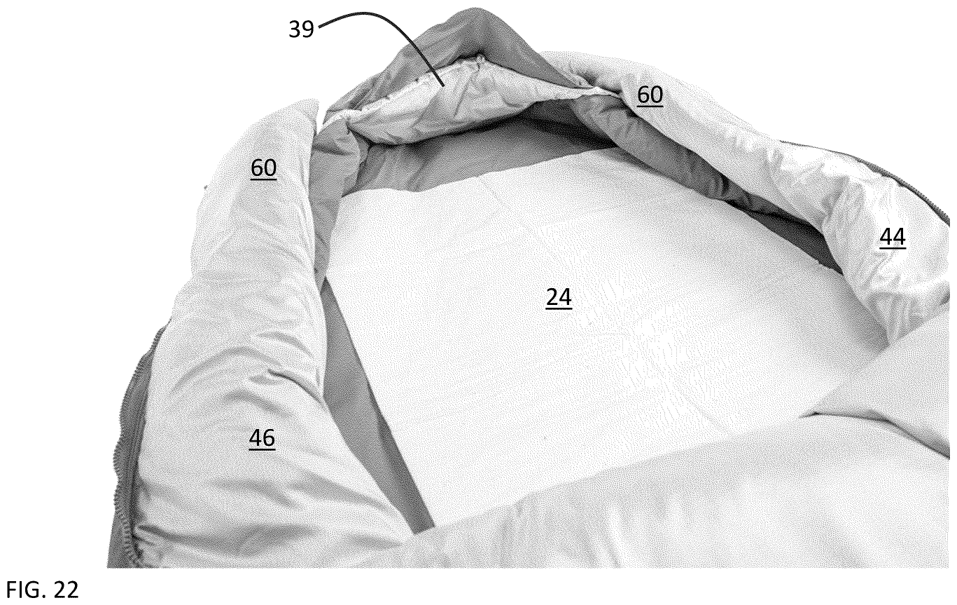

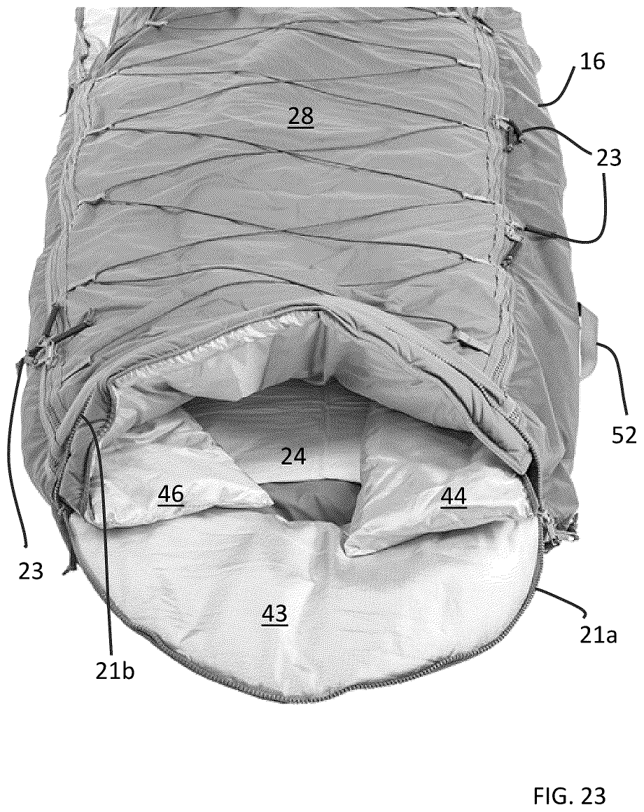

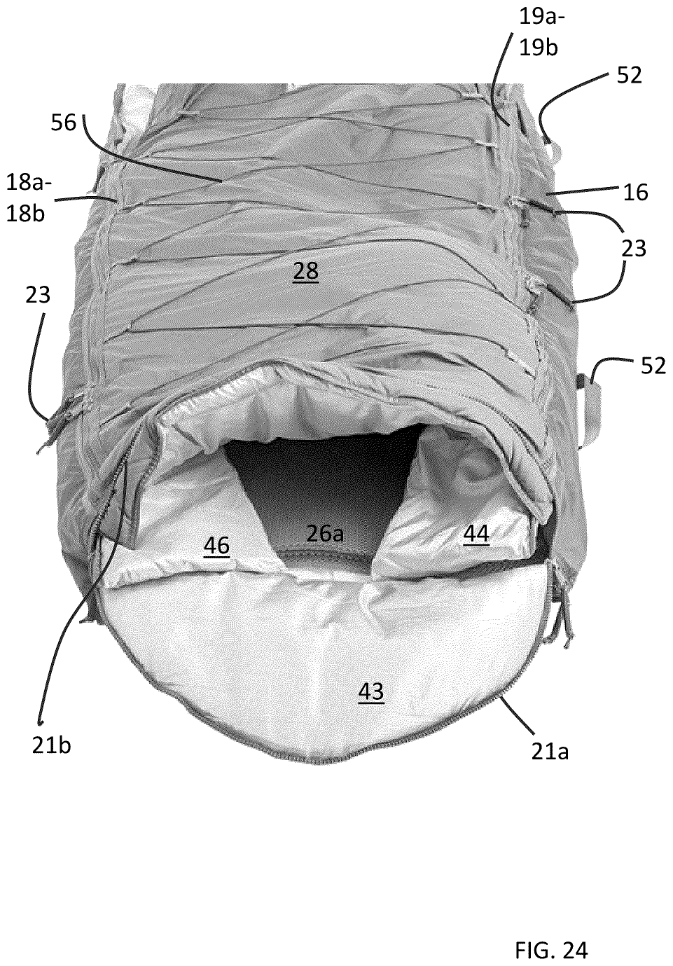

FIGS. 1A-1B present sectional views of a hypothermia survival bag (HSB) 10, according to an embodiment of the invention. The HSB 10 may include two outer materials, an outermost durable water repellent shell 12 and a durable waterproof liner 14 which work together to keep water out of the bottom of the HSB 10, forming a waterproof base 16. Each layer, i.e., water repellent shell 12 and waterproof liner 14, of the waterproof base 16 may be made from a single piece to minimize seams. The HSB's waterproof base 16 is shaped like a bathtub, placing the patient access zippers 18a-18b, 19a-19b, 21a-21b (See FIGS. 2-31) at the upper edge of the waterproof base 16, which is well above the midline around the perimeter of the HSB 10. The bathtub design and elevated arrangement of the zippers 18a-18b, 19a-19b is advantageous in that the elevated zippers make it easier for medical personnel to access the patient inside the HSB, and the elevated zipper makes water, dirt, or snow intrusion less likely when the zipper is used, even when the HSB 10 is placed on the ground. Each of the zippers 18a-18b, 19a-19b, 21a-21b include a plurality of zipper pull pairs 23. Multiple pairs, e.g., 2, 3, or even 5 sets of zipper pull pairs 23 may be included on zippers 18a-18b, 19a-19b, 21a-21b. The multiple pairs of zipper pulls 23 permit multiple points of access to the patient without opening the entire insulated top 28 or exposing the patient more than necessary. This helps to retain the warmth inside the HSB 10.

The waterproof base 16 envelopes a number of layers inside the top, bottom and sides, as illustrated in FIG. 1A. The bottom inside layers may include a batting layer 20 with aerogel, such as Primaloft® CrossCore. The top edges of the waterproof base 16 are joined to a first half of a continuous zipper 18a-18b (right side of HSB 10), 19a-19b (left side of HSB 10), and 21a-21b (foot enclosure 42). The continuous zippers 18a-18b, 19a-19b extend from the hood 39 and head opening 40 down to the foot enclosure 42 and foot panel 43. A durable water repellent shell 22 may be over the batting layer 20. The water repellent shell 22 serves to protect and isolate the batting layer 20 from any liquids inside the HSB 10, including blood, urine, water, and other liquids not captured by the absorbent pad 24. If allowed to infiltrate the batting, the liquids may reduce the insulative qualities of the batting 20. An absorbent pad 24 may be placed on top of the shell 22 to absorb any of the liquids described above. A perforated spacer 26 over the absorbent pad 24 serves to promote liquid drainage away from the patient and separation of the patient from the cooling fluids. FIG. 1B presents a sectional view of the HSB 10 with the various individual layers, 12, 14, 20, 22, 24, 26, 30, 32, 34, 36, 38 that may be grouped into subassemblies. Layers 1-2 collectively correspond to insulated top panel 28. Layers 3-4 collectively correspond to the water repellent shell 12 and the waterproof, insulating base 14, 20, 22 of the HSB. Layer 5 corresponds to the insulated walls 44, 46. Additional descriptions of Layers 1-5 are provided below.

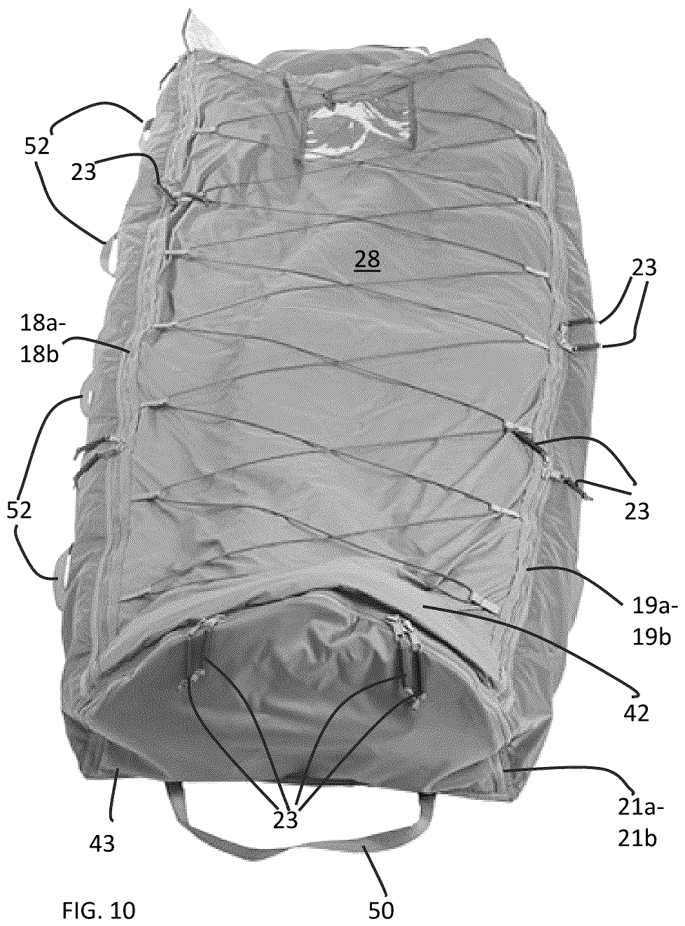

There is a head opening 40 between the zipper's ends for a patient's head, as presented in FIGS. 2-3 and 17-19. A hood 39 is incorporated into the waterproof base 16 and is sized to accommodate a patient's helmet and other head-worn equipment. The zippers 18a-18b, 19a-19b abut the zipper 21a-21b of the foot enclosure 42, as seen in FIGS. 10-12. In another embodiment (see FIG. 32) the zipper 21a-21b is eliminated to simplify the design because medical providers felt they could access the foot enclosure 42 just as easily via zippers 18a-18b and 19a-19b. A foot panel 43 is incorporated in the waterproof base 16 and is sized to accommodate a patient's boots or other footwear.

The fully openable insulated top panel 28 includes several layers of distinct materials. The upper outermost layer of the HSB 10 may include a wind-resistant, water repellent shell 30, such as Brookwood's® Eclipse. Below the shell 30 may be an insulating layer 32, which may be made of two or more layers of batting, such as 3 ounce per square yard of Climashield®; this batting was chosen because it is claimed to have loft recovery after compression. The next layer down is a first metallized shell 34, such as Brookwood's® metallized Balloon, that serves to reflect thermal energy back towards the patient. Below the first metallized shell 34 may be another batting layer with aerogel 36, with a second layer of metallized shell 38 closest to the patient. Each of the layers of the insulated top 28 is attached to the second half of the continuous zippers 18a-18b, 19a-19b, 21a-21b described above.

Inside both sides of the HSB 10 is an insulated wall 44, 46 comprised of 32, 36 and 38 that are attached by sewing or other means to the zippers 18a-18b, 19a-19b. The insulated walls are suspended from their attachments to the zippers 18a-18b, 19a-19b, and are loose at their bottoms.

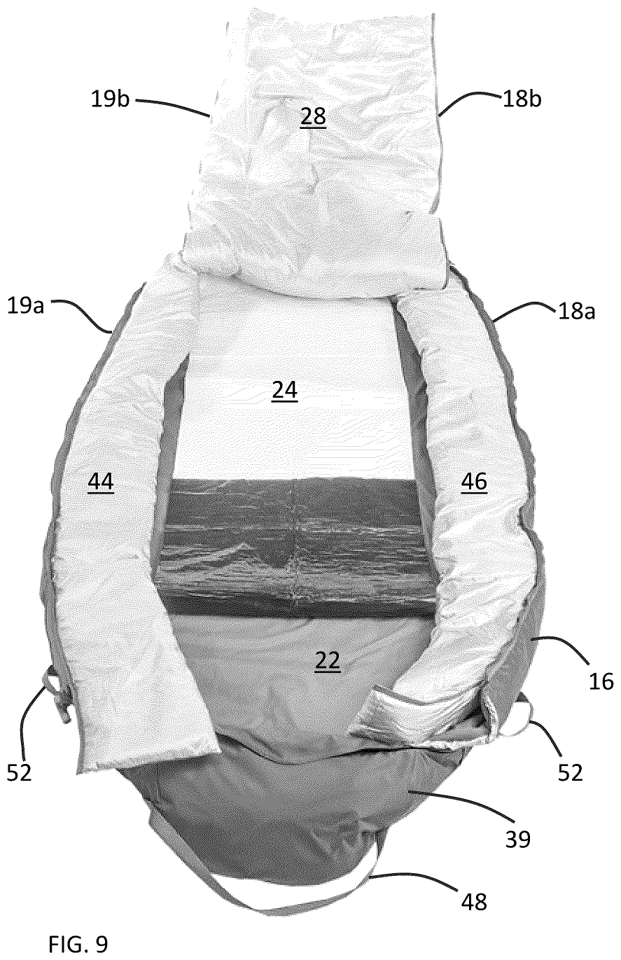

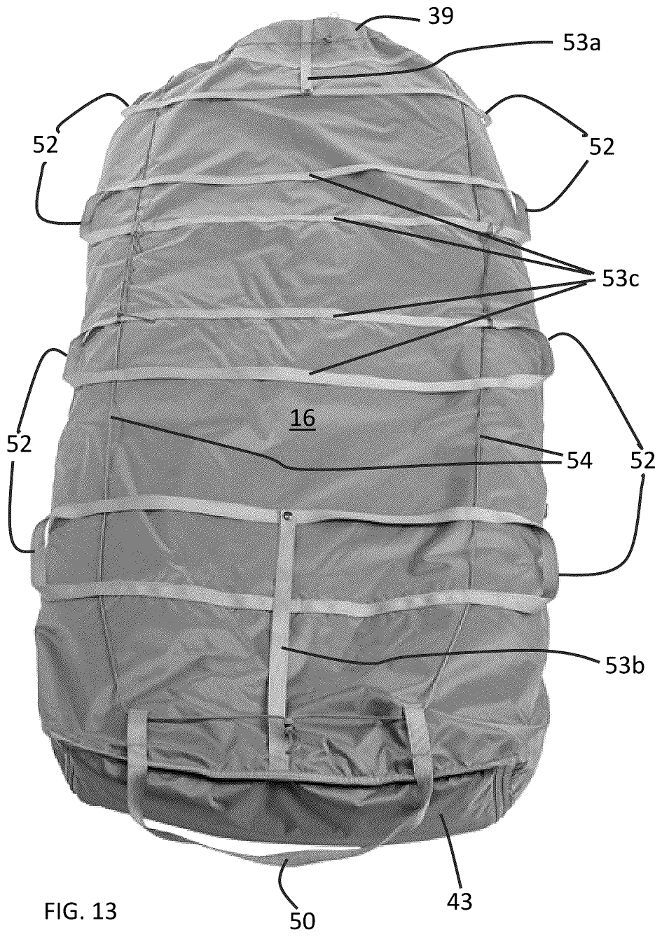

As presented in FIGS. 9-10 and 15, a head transport loop 48 is attached to the bottom outside of the waterproof base 16, and a foot transport loop 50 is attached to a bottom outside of the waterproof base 16. A plurality of side transport loops 52 are presented on both sides of the waterproof base 16, each forming a continuous loop on the waterproof base 16. The straight portions 53c of the transport loops 52 found on the bottom of the HSB (see FIGS. 13-15) are formed to provide an interface that is compatible with stabilizing boards. Every other straight portion 53c floats over (i.e., is not attached to) the waterproof base 16 so that a stabilizing board can slide in-between. As presented in FIGS. 13-15, this embodiment may use a strap at the head 53a and a strap at the foot 53b to secure the stabilizing board, but other variants may have a built-in sleeve or pocket that the board can slide into in order to create a smooth, slick, snag-free bottom. A length-adjustment cord 54 is woven through passages between the head transport loop 48, foot transport loop 50, and side transport loops 52 to gently collapse the HSB 10 to a shorter overall length to better conform to the patient's length. The length-adjustment cord 54 may form a single loop. This is one embodiment for length adjustment.

As presented in FIGS. 2-4, a girth adjustment cord 56 is crisscrossed through loops 58 at the sides of the insulated top 28 to gently collapse the HSB 10 to a smaller overall girth to better conform to a patient. The length adjustment cord 54 and the girth adjustment cord 56 are intended to be used gently to remove excessive void space but without compressing the insulation against the patient inside the HSB 10.

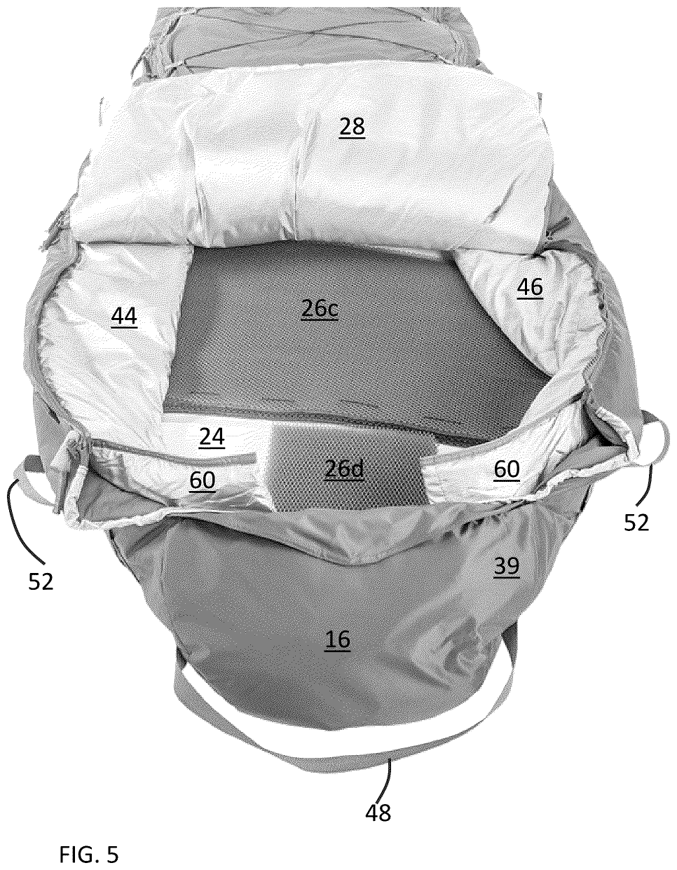

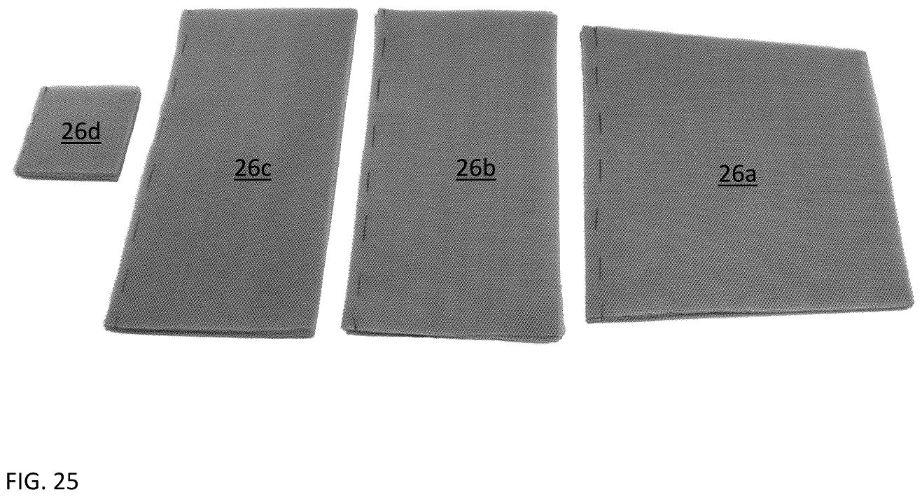

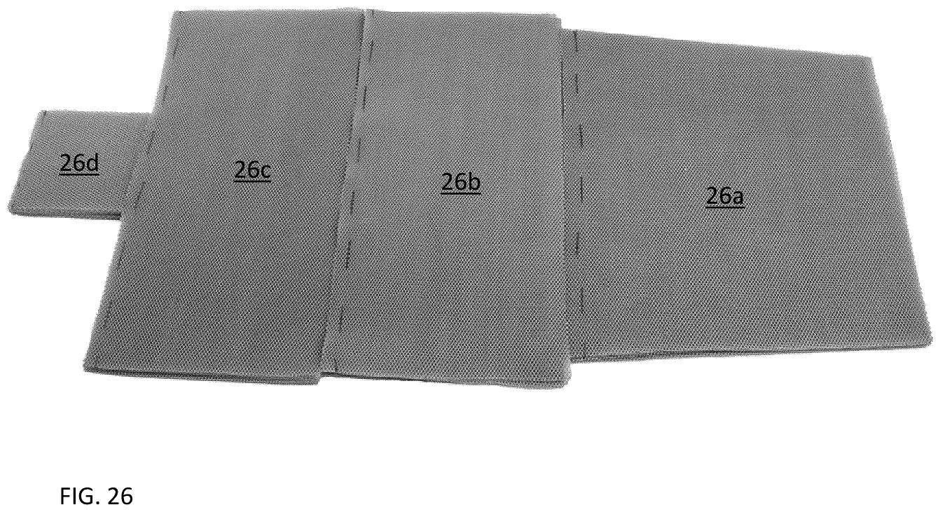

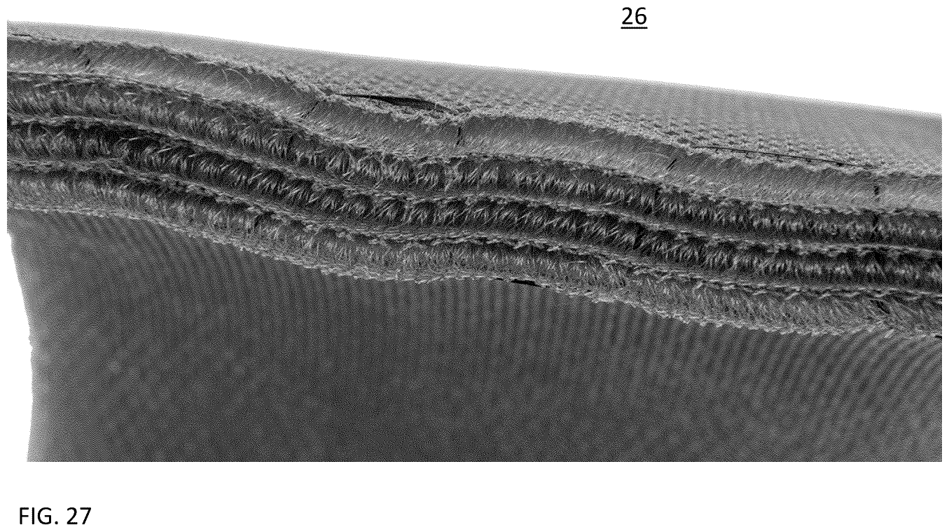

As presented in FIGS. 5-6 and 25-26, the perforated spacer 26 may include a plurality of individual pieces 26a-26d to allow the perforated spacer 26 to adapt to the length of the patient. The individual pieces 26a-26d may be spaced slightly apart (FIG. 25) for longer patients, or closer together or overlapping for shorter patients (FIG. 26). FIG. 27 presents an end view of a spacer pad 26 showing the plurality of layers incorporated into a single perforated spacer 26. The perforated spacer 26 may be a composite of many layers or as few as one layer.

The hypothermic patient is placed inside the HSB 10, where he is surrounded by the layered insulation described above. While inside the HSB 10, the patient may lay atop a stack of materials that insulate and protect, providing drainage and isolation from fluids, if the medical situation warrants it. As presented above (see FIGS. 1A-1B), the general material layup arrangement is designed like a body-mapped, layered clothing system. The layered system applies to all sides of the HSB, but the types of layers, e.g., the combinations of materials, used on a given side depends on the desired functionality that is needed in that part of the HSB. Numerous options are possible. In general terms, the layer closest to the patient (Layer 1, Layer 3, Layer 5) may be an insulator comprised of shell materials that sandwich an insulating material; each of the Layer 1, Layer 3, and Layer 5 embodiments may incorporate aerogel, which has been proven to provide good insulation. The material layup of the next layer or two out depends on their location within the HSB. In all cases, the outermost layer provides some manner of environmental protection.

The top panel 28 may have a two-layered system. Layer 1 enables the HSB to heat up quickly, to insulate, and to allow internal moisture vapor to escape to Layer 2; Layer 1 of the top panel 28 is comprised of a metalized shell 38 plus a nonwoven batting with aerogel 36 plus a metalized shell 34. Layer 1 of the top panel 28 is intentionally thinner than Layer 2 of the top panel 28, so that it heats up quickly when occupied; reflected body heat and an external heating source (e.g., chemical heater, electric heating panel) would only speed up the warming process.

Layer 2 of the top panel 28 enables the HSB to insulate, to recover loft quickly after being packed, to provide protection from wind and light precipitation, and to allow internal moisture vapor to escape the HSB; the latter is important to prevent further cooling of the hypothermic patient. Layer 2 of the top panel 28 is comprised of a metalized shell 34 (that is shared with Layer 1) plus two layers of a nonwoven batting 32 plus a wind-resistant, water repellent shell 30. Layer 2, the outer layer of the top panel 28, intentionally uses two layers of thinner nonwoven batting to mitigate the issue of “thin spots,” which are common during the production of heavier weight nonwoven battings. In addition, lighter weight batting production appears to produce a more consistent, uniform product that yields more consistent insulation performance.

The bottom of the HSB 10 has a layered system that is designed to insulate the patient while keeping out environmental moisture (Layer 3) and to enable patient carriage/transport (Layer 4). Layer 3 of the HSB bottom may be a shell-enclosed insulation that is comprised of a water repellant shell 22 plus a nonwoven batting with aerogel 20 plus a waterproof shell 14. Layer 4 of the HSB bottom includes the water repellant shell 12.

The sides of the HSB have a layered system (Layers 3, 4, 5). The layered system is designed to insulate the patient (Layers 5 and 3), to provide structure to the HSB that enables elevated patient access (Layer 5), to recover loft quickly after being packed (Layer 5), and to keep the environment out (Layer 4). Layer 5 of the HSB's sides is a shell-enclosed insulating hung liner that is formed from a single piece of metallized shell 38 that envelopes a nonwoven batting with aerogel 36 and a nonwoven batting 32. Additional insulation is provided by Layer 3 which is comprised of a water repellant shell 22 plus a nonwoven batting with aerogel 20 plus a waterproof shell 14. Layer 4 of the HSB's sides is the water repellant shell 12.

The sides of the HSB 10 use a durable waterproof liner 14. Despite the illustration of FIG. 1, the waterproof liner 14 is a single continuous, unperforated piece of fabric that creates a waterproof bathtub 16 for the patient. This is critical for multiple reasons: (1) water is 25× more conductive than air, so any liquid contacting the patient will cool them further; (2) the medical provider may not have protected or dry options for placing the HSB 10 when treating the patient, especially during periods of rain or thaw. The spacer pad 26 augments this capability and isolates the patient from the absorbent pad 24.

The patient material stack is comprised of materials that insulate and protect the patient from below. (Note that some of these materials are optional based on the type of rescue.) As described above, the patient sits atop a porous, spacer material 26 that allows any fluids coming off of or out of the patient (e.g., water, melting snow, urine, blood) to collect in the absorbent pad 24 below. The porous spacer 26 also creates a physical separation between the patient and the fluids, which prevents them from being further cooled by these fluids. Below the absorbent pad 24, which is a pad that can absorb up to 3-liters of fluid, is a water repellent shell 22 that makes up the interior floor of the HSB 10. Aerogel insulation 20 is sandwiched between the water repellent shell 22 and the waterproof liner 14 to keep it dry. The durable water repellent shell 12, such as Brookwood® Yuma, makes up the very bottom of the HSB 10; patient carrying handles 48, 50, 52 are attached only to this fabric 12 so that the waterproof liner 14, such as Brookwood® Yuma HST, remains intact, i.e., unperforated.

PROBLEM—Quickly Heating the Patient.

This problem is addressed, in part, using the material layering described in the previous sections. The full-opening insulated top 28 allows for rapid emplacement of the hypothermic patient. Research has shown that proper garment fit is crucial to insulation. If an insulated garment is too large, then it will not insulate the wearer very well. This is true for the HSB 10, which is why it may have the girth and length adjustment features, described herein.

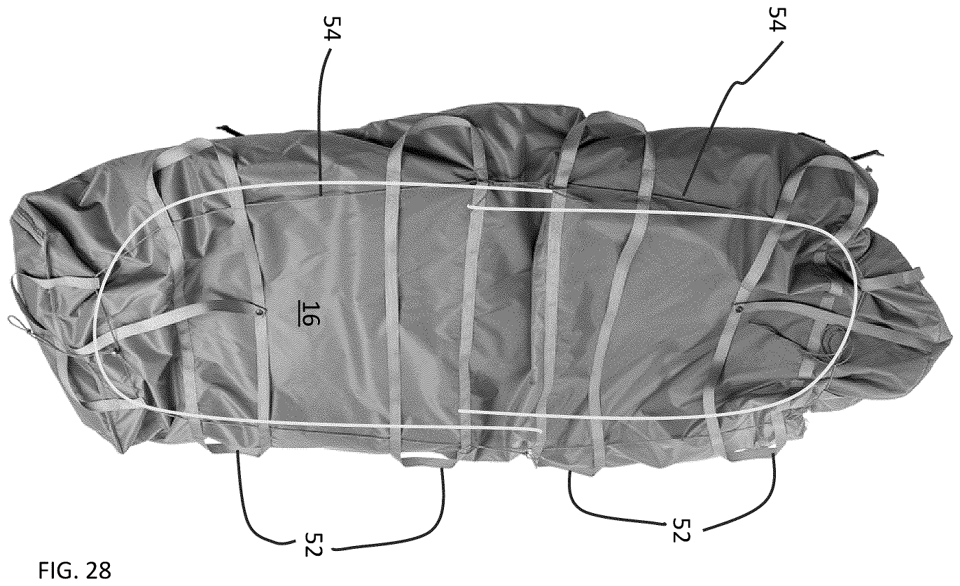

Length adjustment loops—These loops allow the medical provider to pre-string 550 cord 54 or similar to shorten the overall length of the HSB 10. Likewise, the medical provider may simply fold over the foot 42 of the HSB 10 to shorten its overall length. In one embodiment, the back side of the HSB may include two pre-strung cords 54 that may be used to selectively shorten the bottom half and/or top half of the bag. See FIG. 28. The cords 54 may be strung through spaces between the waterproof base 16 and the side transport loops, foot transport loop 50, and head transport loop 48. These cords 54 may be omitted if they are determined to be a snag hazard. The general arrangement of the length adjustment cords 54 are emphasized in FIG. 28 with the drawn lines superimposed on the photograph.

To facilitate length adjustment of the HSB 10, the porous spacer panel 26 may be cut into four segments: head 26d, torso 26c, buttocks 26b, and lower leg 26a. The spacer panels 26a-26d may be pushed together or overlapped to reduce overall length (see FIG. 26).

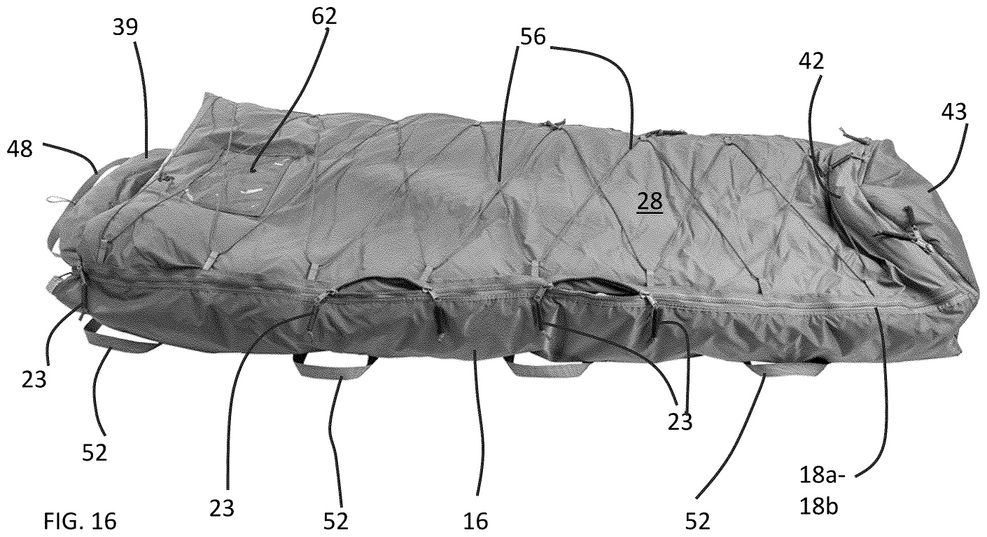

Girth adjustment—This adjustment mechanism enables the medical provider to quickly snug the HSB 10 around the patient, without being too tight. The girth adjustment mechanism 56, 58 (see FIGS. 16-18, for example) runs the full length of the bag, but it allows for variable adjustment along the length of the HSB 10. For instance, the torso may require less cinching than the legs; the adjustment mechanism allows for this.

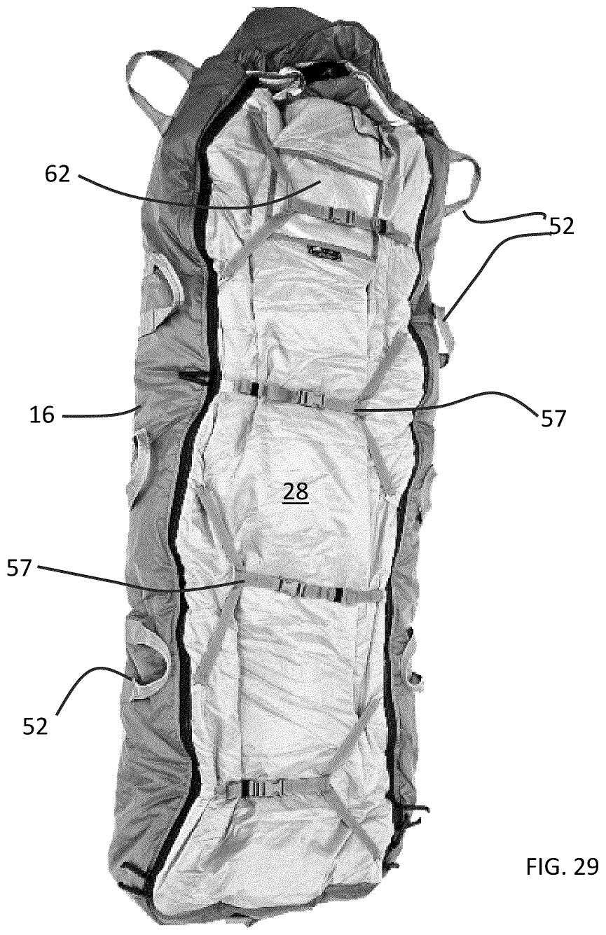

The mechanism may be a corset style as presented in FIGS. 16-18, crisscrossing cord; adjustment may easily be made by grabbing an “X,” pulling the cord 56 and tying a knot. Multiple knots may be made, if required. An alternative, simplified approach may be made using multiple Y-shaped cinches 57 (see FIGS. 29, 30, and 32), which may be similar to the adjustment straps on a backpack. Advantageously, the girth adjustment mechanism 56, 58 is specifically limited to the top cover 28 to keep the patient access route clear.

The adjustable sizing of the HSB 10 enables life-saving equipment like heaters and oxygen tanks to be stably packed in the HSB with the patient.

PROBLEM—Retaining heat is difficult, especially while monitoring and accessing the patient. Trapping heat is crucial to the survivability of a hypothermic patient, but the patient cannot be stuffed in a bag and left alone. They must be monitored and accessed from time to time. The HSB enables this in a number of ways.

Adjustable foot access and adjustable patient access is achieved using multiple zipper pairs 23 that are easily moved along the footbed (zipper 21a-21b) or both sides of the body (zippers 18a-18b, 19a-19b) to access the body. A zipper pair 23 may be slid into place and opened at precisely the location of a wound. This precision opening from an elevated position enables the medical provider to do their job without severely impacting heat retention and jeopardizing the patient. Some of the zipper pairs 23 may be used for equipment access lines, e.g., power, warm fluids.

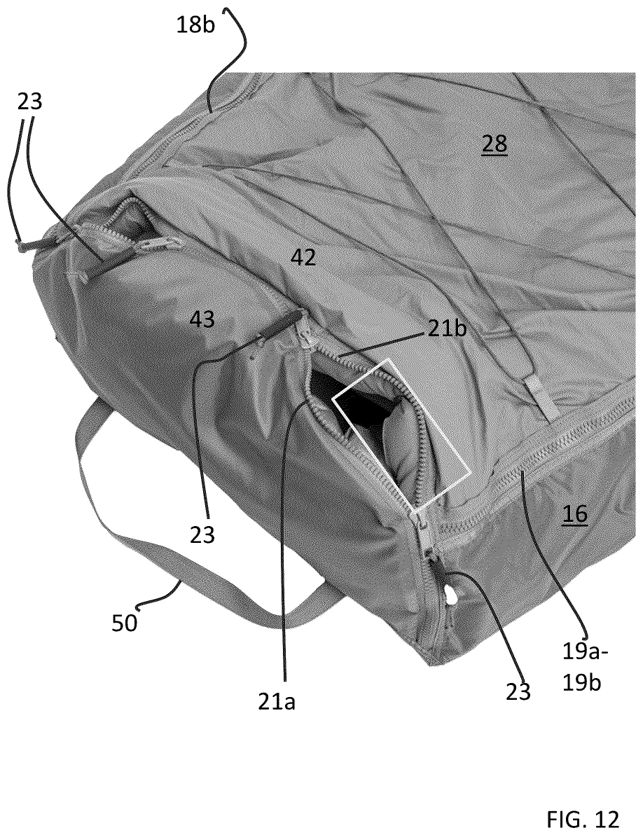

The zippers 18a-18b, 19a-19b, 21a-21b are windproof and an insulated flap is behind each one to help to ensure that heat does not escape when the zippers are closed. The insulated flap may be seen inside the box on FIG. 12. This flap closes the gap behind the zipper.

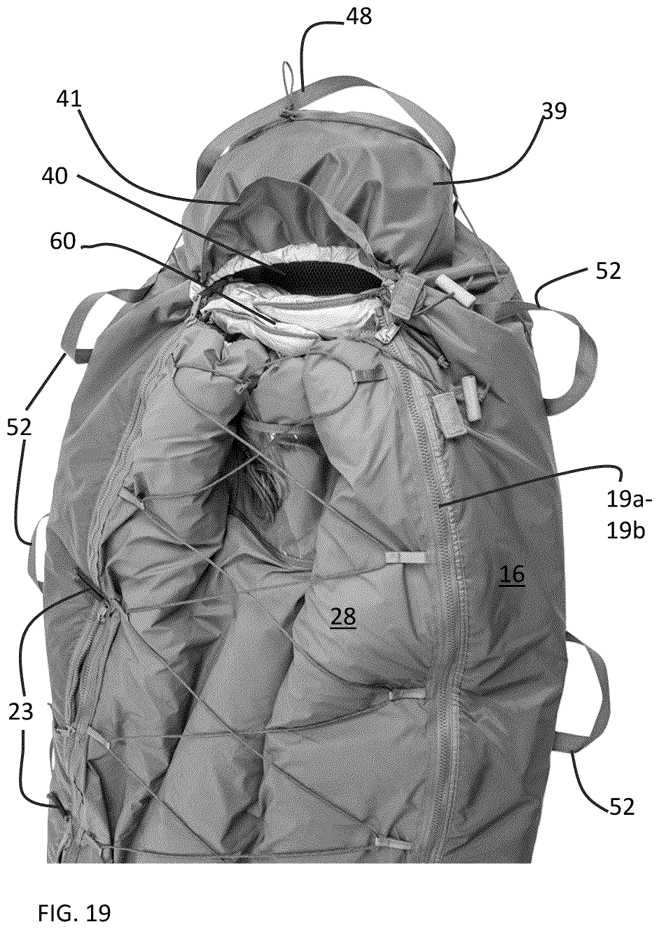

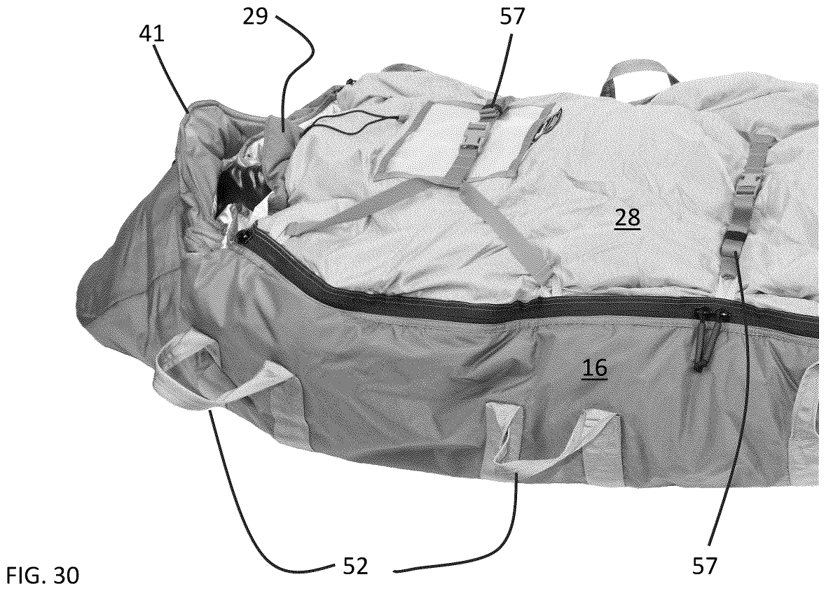

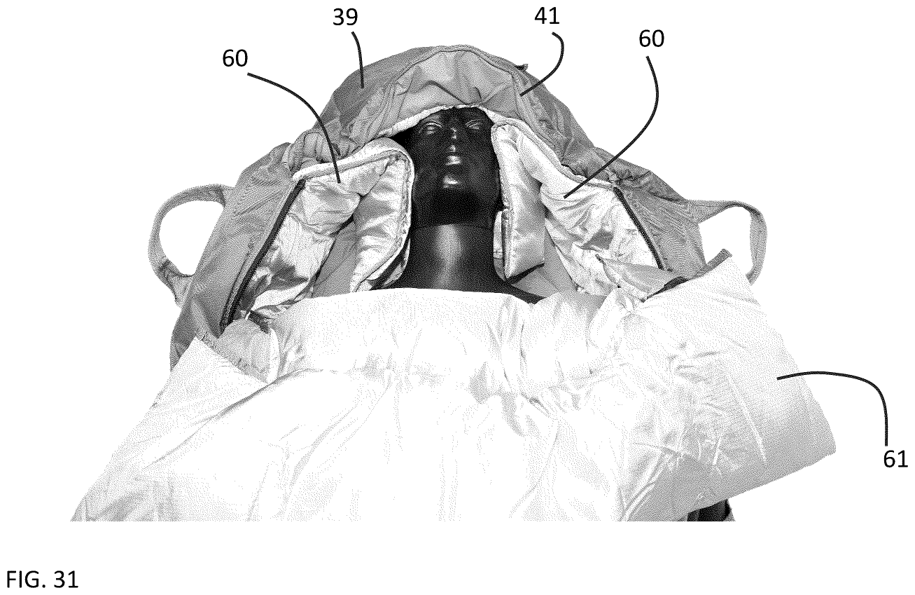

The insulated hood 39 cinches down to conform to the patient's head. The insulated hood 39 with flexible brim 41 helps to protect the face from the elements and to trap the heat around the patient's head. The hood 39 and top panel 28 both have flexible extensions that create brims 41 or duck bills 29 (see FIGS. 31-32); only the hood brim 41 is shown in the FIG. 19 variant, while both brims are shown in the FIG. 30 variant. Together, the upper and lower duck bills 29, 41 create a micro-environment and a small opening that serves multiples purposes—it enables patient observation/communication with minimal heat loss; and it helps to heat the air the patient inhales, especially when lined with fur (not shown). The hood opening 40 may be cinched around the patient's face, if desired. The hood 39 is large enough to accommodate a helmet and neck brace in the event of a head or neck injury.

The insulation extensions 60 inside the HSB 10 help provide local insulation. The neck insulation 60 is effectively an extension of the hung insulated walls 44 and 46. The neck wrap insulation 60 (see FIGS. 5, 6, and 31) helps to insulate the patient's neck and keep heat from escaping. These extensions may be wrapped around the patient's neck and/or stuffed into the hood 39 and/or wrapped around the patient's hands to provide local insulation. The chest extension 61 is similar to the neck insulation 60. The chest extension 61 extends from the insulated top panel 28 and provides extra insulation around the patient's face and creates a thermal barrier to prevent heat leakage from the opening of the HSB. The footbed insulation extensions (similar to the neck insulation 60) may be wrapped around the feet and toes to provide added local warmth.

The patient card pouch 62 (FIGS. 2 and 29) enables patient information to remain visible and to stay with the patient.

PROBLEM—Accessing the patient without snow, water, dirt or debris getting inside.

Current hypothermia kits are secured (via zipper or hook and loop) along the ground. This is problematic for the following reasons—(1) it forces the medical provider to open wide the hypothermia kit when accessing the patient, which causes significant heat loss; (2) it has the great potential to bring water, rain, snow or ice into the hypothermia kit, that contacts the patient or their clothing and cools the patient down; and (3) it has the great potential to bring dirt, sand, vegetation or debris into the hypothermia kit that houses a patient who may have open wounds. The HSB 10 solves this problem by elevating patient access (zippers 18a-18b, 19a, 19b) to the upper sides of the body. The HSB 10 does this by creating hung insulated walls 44 and 46 inside a bathtub shaped waterproof base 16.

PROBLEM—Accommodate a wide range of patient sizes.

Based on field work, the HSB 10 can comfortably accommodate a 320 lb, 76-inch tall male. The porous spacer 26a-26d may be pushed together to accommodate a 5′2″ patient or expanded to accommodate a 6′4″ patient.

PROBLEM—Heating the patient while allowing moisture to escape.

The patient may be ultimately heated by placing a heat source (not shown) on or around the patient in the HSB 10 and allowing that heat to drive its way into the patient's body. Heat sources may include chemical heaters or a battery-powered, electric heating panel. The HSB 10 is compatible with these heat sources. The material layup, specifically the vapor permeable shells 30, 34, and 38 that comprise the top panel 28, allow moisture to escape rather than accumulate.

PROBLEM—Elevating the patient so that they aren't losing heat to the ground and laying in run-off fluids that continue to cool the patient.

The HSB 10 solves this problem with the porous spacer fabric 26 and full-length absorbent liner 24. Additional information about these features can be found in the text below, in the material layup description.

The patient material stack is comprised of materials that insulate and protect. Note that some of these materials are optional based on the type of rescue. First, the patient is placed atop a porous, spacer material 26 that allows any fluids coming off of or out of the patient, e.g., water, melting snow, urine, blood, to collect in the absorbent pad 24 below. The porous spacer also creates a physical separation between the patient and the fluids, which prevents the patient from being further cooled by these fluids. Below the absorbent pad 24, which is a commercially-available pad that can absorb up to 3-liters of fluid, is a water repellent shell 22 that makes up the interior floor of the HSB 10. The aerogel insulation 20 is sandwiched between the water repellent shell 22 and the waterproof liner 14 to keep it dry. Aerogel is selected because it is a good insulator that reduces heat loss to the ground. The durable water repellent shell 12 makes up the very bottom of the HSB 10; patient carrying handles 48, 50, 52 are attached to the water repellent shell 12 so that the waterproof liner 14 above it remains intact, i.e., unperforated.

PROBLEM—Being able to move the patient once he is stabilized in the HSB.

The outermost layer (shell 12) of the HSB 10 has webbing straps 48, 50, 52 affixed to it to enable patient lift and transport while safely packaged inside the HSB 10. There may be a total of ten patient transport loops-four on the right, four on the left, one at the head and one at the feet, as presented in FIGS. 2-15. The loops are large enough to accommodate a gloved or mitted hand. The construction and spacing of the loops can safely and comfortably support a 320-lb 76-inch patient during a 4-man lift. Comfort is based on keeping the patient from bending at the waist during a lift.

The HSB 10 is compatible with a rescue basket and a stabilizing board (not shown). Other variants of the HSB 10 may have a pouch option that enables the stabilizing board to be slipped into place underneath the HSB-emplaced patient.

Other variants of the HSB 10 may have a slick, i.e., feature-reduced, bottom (shell 12) to facilitate reduced-resistance dragging of the patient in the HSB 10.

PROBLEM—Reuse.

The HSB is comprised of common materials found in garments. As a result, the HSB, except for the absorbent pad 24 may be laundered and reused.

PROBLEM—Make it lightweight and packable.

The HSB 10 may be collapsed and packed into a backpack. The HSB 10, without absorbent liner 24 and porous spacer 26 weighs about 10 lbs. A porous spacer 26 and absorbent liner 24 may bring the weight up to about 15 lbs. This advantage is particularly suitable when a snowmachine or rotary wing aircraft is used for transport.

PROBLEM—Being able to see the patient and medical provider from a hovering rotary wing aircraft, such as during civilian search and rescue.

The HSB 10 may be made with an insulated top 28 comprising a bright cover shell, e.g., yellow, with the same properties described above, and with bright girth adjustment straps 57, e.g., red (see FIGS. 29-32). A dedicated high-visibility storage backpack may be included with the HSB 10.

A ‘pocket’ may be formed in the bottom of the HSB 10, on the external surface of the waterproof base 16 by attaching the side transport loops 52 only at the sides of the durable water repellant shell 14. If required, a stabilizing board (not shown) may be placed into the pocket. The hypothermic patient may then be placed into the Hypothermia Survival Bag 10. Heating and insulating panels are emplaced, and the bag is zippered shut. The HSB 10 is then adjusted to accommodate the head, height, and girth. Any fluids coming off the body flow through the spacer panel 26a-26d into the absorbent pad 24 or liner. The patient is accessible along a top edge of the HSB 10 using zipper pairs 23 along the body. The patient may be moved using the transport loops for lifting.

Numerous options and configurations are possible, depending on the needs of the user. Such variations and configurations may include simplified girth adjustment, a smooth bottom, alternate cover color, dedicated pocket for a stabilizing board, removal of foot access, lengthened neck insulation, the addition of a flexible chest brim, and/or refined spacer fabric.

This written description uses examples as part of the disclosure, including the best mode, and also to enable any person skilled in the art to practice the disclosed implementations, including making and using any devices or systems and performing any incorporated methods.

The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. The embodiments disclosed herein are not intended to unnecessarily limit the scope of the disclosure. Therefore, simple modifications or variations belonging to the equivalent of the scope of the claims and the instructions disclosed herein for a patent are all within the scope of the present disclosure.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any nonclaimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Components and features described herein may be combined in any desired manner to achieve the desired performance goals.

1. A hypothermia survival bag comprises:
a waterproof base further comprising a water repellent shell outer layer and a waterproof liner, wherein the waterproof base has a bathtub configuration having a head end, a foot end, and left and right sides;
an insulated top panel attached to the waterproof base at the foot end, and removably attached to each of the left and right sides with left and right side zippers, wherein the left and right side zippers extend from the foot end to the head end, wherein each of the left and right side zippers includes a plurality of pulls permitting each of the left and right side zippers to be selectively opened in multiple places simultaneously, wherein the left and right zippers are at an upper edge of the bathtub configuration to provide elevated access;
a foot box;
a hood having a brim; and
a patient card pouch attached to an outside of the insulated top panel.

2. The hypothermia survival bag of claim 1, further comprising
a plurality of side transport loops, a head transport loop, and a foot transport loop attached to the water repellent shell of the waterproof base.

3. The hypothermia survival bag of claim 2, wherein the plurality of side transport loops, the head transport loop, and the foot transport loop are selectively attached to the water repellent shell of the waterproof base in order to accommodate a stabilizing board between the plurality of transport loops and the water repellent shell of the waterproof base.

4. The hypothermia survival bag of claim 1, wherein the insulated top panel includes a duck bill extension at the head end.

5. The hypothermia survival bag of claim 1, wherein the bottom interior of the waterproof base includes a plurality of insulating layers selected from the group consisting of batting with aerogel, a durable water repellent shell, an absorbent pad, and a perforated spacer pad.

6. The hypothermia survival bag of claim 5, wherein each of the interior left and right sides of the waterproof base comprise an insulating curtain attached adjacent the left and right side zippers, the insulating curtain hanging down to the bottom interior of the waterproof base.

7. The hypothermia survival bag of claim 6, wherein each of the left and right insulating curtains includes a plurality of insulating layers selected from the group consisting of a batting layer, a batting layer with aerogel, and a metallized shell.

8. The hypothermia survival bag of claim 1, wherein the insulated top panel includes a plurality of functional and insulating layers selected from the group consisting of batting with aerogel, a wind-resistant shell, and a metallized shell.

9. The hypothermia survival bag of claim 6, wherein the insulating curtains further comprise insulating extensions for the neck and feet.

10. The hypothermia survival bag of claim 1, further comprising a girth adjustment mechanism attached to left and right edges of the insulated top panel, the girth adjustment mechanism selected from Y-straps with buckles and a girth adjustment cord laced through eyelets along the left and right edges of the insulated top panel.

11. The hypothermia survival bag of claim 1, further comprising a length adjustment mechanism incorporated into a bottom exterior of the waterproof base, the length adjustment mechanism comprising one or more length adjustment cords laced through openings in the side transport loops, the head transport loop, and the foot transport loop.
